(19) United States
(12) Patent Application Publication
(10) Pub. No.: US 2026/0308085 A1
(43) Pub. Date: Oct. 8, 2026

(54) OXIDATIVE COUPLING OF METHANE IN HYSTERESIS CONDITION

(71) Applicant: Braskem S.A., Camaçari (BR)

(72) Inventors: Normando José Castro de Jesus, São Paulo City (BR); Tahyna Barbalho Fontoura, São Paulo City (BR); José Carlos Pinto, São Paulo City (BR); Henrique Pacheco, São Paulo City (BR); João Victor Marques de Queiroz, São Paulo City (BR); Vitor Duarte Lage, São Paulo City (BR)

(73) Assignee: Braskem S.A., Camaçari (BR)

(21) Appl. No.: 19/542,502

(22) Filed: Feb. 17, 2026

Related U.S. Application Data
(60) Provisional application No. 63/759,343, filed on Feb. 17, 2025.

(51) Int. Cl. C07C 2/84; B01J 8/02; B01J 23/10; B01J 23/34; B01J 35/40

(52) U.S. Cl. CPC C07C 2/84; B01J 8/0278; B01J 23/10; B01J 23/34; B01J 35/40; C07C 2523/10; C07C 2523/28; C07C 2523/30

(57) ABSTRACT

A system can perform catalytic oxidative coupling of methane, where the system includes: a fixed-bed reactor that operates at adiabatic condition and receives a stream of oxygen and methane; and a catalyst present inside of the fixed-bed reactor that performs catalytic oxidative coupling of methane in a hysteresis mode, where the catalyst is in the form of pellets or powders with average particle size ranging from 50 μm and 40 mm, and has a morphological shape of hollow, spheric, multilobe, and others, and a ratio between an external surface area (A) and an external volume (V) in the range of 100 m⁻¹ and 30000 m⁻¹.

### CROSS REFERENCE TO RELATED APPLICATIONS

The present application claims the benefit of U.S. Provisional Patent Application U.S. 63/759,343, filed on Feb. 17, 2025.

### FIELD OF THE INVENTION

This invention generally relates to the field of oxidative coupling of methane processes, which uses methane from flue gas of a liquid or gas cracker or methane from a renewable as a low-cost feedstock, or commercial grade natural gas, to produce, mainly, ethylene with lower levels of CO₂ emissions.

### BACKGROUND OF THE INVENTION

The oxidative coupling of methane (OCM) is a promising process for converting methane into higher hydrocarbons, particularly ethylene, which is a valuable chemical feedstock. The process involves the reaction of methane (CH₄) with oxygen (O₂) at high temperatures to produce ethylene (C₂H₄) and other C₂+ hydrocarbons. For that purpose, methane from flue gas of a cracker or methane from a renewable source or natural gas could be used as feedstock to produce, mainly, ethylene with lower levels of CO₂ emissions than the current cracker technology.

Oxidative coupling of methane has been studied for a long time; however, no commercial unit has been deployed. Most of the development focuses on different catalysts, reactor designs or theoretical studies on hysteresis. The known processes show low conversion (~10%), leading to high recycling rates and, in consequence, high CAPEX, prejudicing the economic feasibility.

Among such studies, it can be cited document CA2874043A1, which discusses the use of monolithic catalysts and mentions hysteresis in the catalyst description. The focus is on the formulation and structure of catalysts used in OCM processes.

Document “Autothermal Methane Oxidative Coupling Process over La₂O₃/MgO Catalysts” by Andrei L. Tarasov and Leonid M. Kustov is an experimental study explores the use of La₂O₃/MgO catalysts in the OCM process. The research highlights the efficiency and performance of these catalysts in promoting the oxidative coupling reaction.

Document “Autothermal Oxidative Coupling of Methane with Ambient Feed Temperature” by Sagar Sarsani, David West, Wugeng Liang, Vemuri Balakotaiah is a theoretical study that investigates the OCM process at ambient feed temperatures. The research aims to optimize the process conditions to achieve higher yields of ethylene and other C₂+ hydrocarbons.

These documents collectively contribute to the understanding and advancement of OCM processes, highlighting the importance of catalyst formulation, reaction conditions, and reactor design in achieving efficient and effective methane conversion.

There thus remains a continuous need in the art for development of more feasible processes, so as to enhance the conversion of the process with high potential to reduce the capital costs.

### SUMMARY OF THE INVENTION

In one aspect, provided herein is a system for catalytic oxidative coupling of methane comprising: a fixed-bed reactor configured to operate at adiabatic condition and receive a stream comprising oxygen and methane; and a catalyst contained inside of the fixed-bed reactor and configured to catalyze oxidative coupling of the methane in a hysteresis mode, wherein the catalyst is in the form of pellets or powders with average particle size ranging from 50 μm and 40 mm, and has a morphological shape selected among cylindric, hollow, spheric, multilobe and others, and a ratio between an external surface area (A) and an external volume (V) in the range of 100 m⁻¹ and 30000 m⁻¹.

In another aspect, provided herein is a method for catalytic oxidative coupling of methane comprising directing a stream comprising oxygen and methane, mixed upstream of a fixed-bed reactor containing a catalyst; directing two separated streams comprising oxygen to a first inlet and methane to the second inlet of a fixed-bed reactor; catalyzing oxidative coupling of methane in a hysteresis mode; and self-controlling a transfer of heat and mass between the fixed-bed reactor and surroundings by catalyst design.

Additional aspects, advantages and features of the invention are set forth in this specification, and in part will become apparent to those skilled in the art on examination of the following, or may be learned by practice of the invention. The inventions disclosed in this application are not limited to any particular set of or combination of aspects, advantages and features. It is contemplated that various combinations of the stated aspects, advantages and features make up the inventions disclosed in this application.

### BRIEF DESCRIPTION OF THE DRAWINGS

FIG. 1 is a schematic illustration of the system according to the present invention, wherein methane is converted to ethylene and byproducts in a fixed-bed reactor.

FIG. 2 is a schematic representation of the hysteresis behavior in an oxidative coupling of methane.

FIG. 3 is a graph illustrating some conditions obtained by an experimental trial in laboratory in an adiabatic fixed-bed reactor.

FIG. 4 is a graph illustrating the effects of the catalyst particle size on the system and method according to the present invention.

FIG. 5 shows hysteresis behavior of catalyst pellets, comparable to that of powdered catalyst with particle size dₚ>250 μm.

FIG. 6 shows conversion results for a powder catalyst with dₚ<125 μm.

FIG. 7 shows conversion results for a pellet catalyst with dₚ<125 μm.

FIG. 8 shows selectivity results for a powder catalyst (dₚ<125 μm).

FIG. 9 shows selectivity results for a pellet catalyst (dₚ<125 μm).

FIG. 10 is a graph illustrating the effects of catalyst type on the system and method according to the present invention.

### DETAILED DESCRIPTION OF THE INVENTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and may be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein may be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As also used herein, “oxidative coupling of methane” (OCM) is to be generally understood as a chemical route that converts methane into higher hydrocarbons, such as ethylene and ethane, as a way to utilize natural gas more efficiently and produce valuable chemicals.

The disclosure provides a system for catalytic oxidative coupling of methane comprising a fixed-bed reactor configured to operate at adiabatic condition and receive a stream comprising oxygen and methane; and a catalyst contained inside of the fixed-bed reactor and configured to catalyze oxidative coupling of the methane in a hysteresis mode.

According to the present invention, the stream comprising oxygen and methane may be a gaseous mixture comprising oxygen, methane and optionally other components. For instance, such stream may be a mixture of air and methane. The minimum oxygen-to-methane mass ratio required to achieve no hysteresis (with a temperature rise of 400° C. between the reactor feed point and the reactor outlet due to reaction exothermicity) and 100% selectivity for ethane is approximately 0.05. Increasing the mass ratio results in more hysteresis and reduced selectivity.

According to one aspect of the present invention, the system comprises a fixed-bed reactor working at adiabatic condition, which allows a simple and cheap reactor design. Fixed-bed reactors are a type of chemical reactor where the catalyst is fixed in place while the reactants flow over it, which are commonly used in industrial processes due to their efficiency and ability to handle large volumes.

According to another aspect, the fixed-bed reactor is filled with catalysts designed to be sufficiently active and simultaneously provide restricted heat transfer area, in order to limit the rate of heat release from the solid phase to the gas phase, allowing the occurrence of hysteresis behavior.

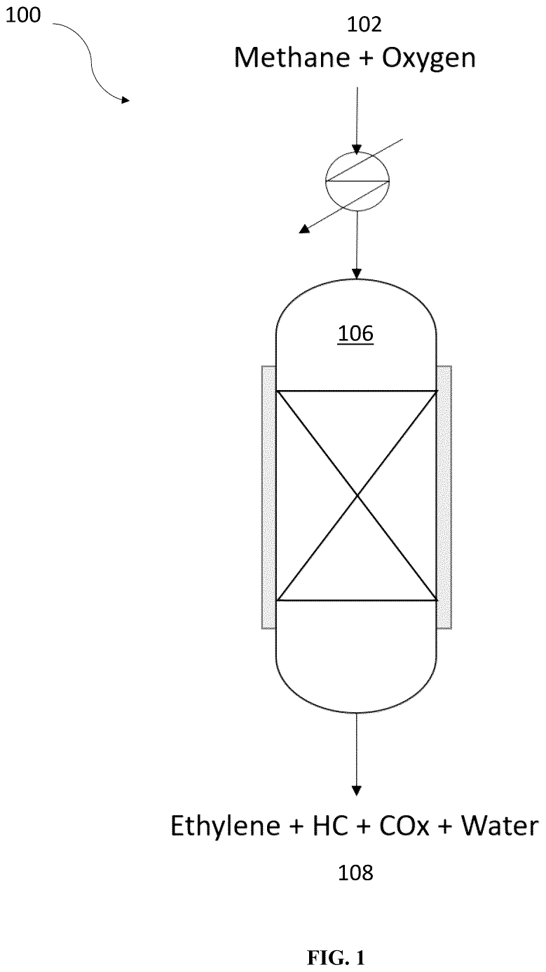

Generally, the OCM process is somewhat similar to the traditional steam cracker processes as per the process sequence, which includes the following steps: (i) reaction, (ii) effluent cooling, (iii) effluent compression, (iv) acid and water removal, (v) cryogenic separation of hydrogen and methane, and (vi) cool separation of light 2-carbon atom compounds from heavier compounds that contain three or more carbon atoms. Despite the similarity of the process sequence, the yields can be very different depending on several features of the process and the catalyst employed, thus resulting in advantageous differences in equipment size and operation unit design. The system 100 according to the present invention considers the reactor design as depicted by FIG. 1, which comprises a fixed bed reactor 106 configured to operate at autothermal adiabatic condition and to receive a stream comprising oxygen and methane 102. The fixed-bed reactor 106 is filled with catalyst specifically designed to promote oxidative coupling of methane reaction in a hysteresis behavior. As result, the outlet stream 108 contains mainly ethylene and other hydrocarbons, hydrogen, non-converted methane, other hydrocarbons, COₓ and water.

In accordance with an embodiment, the fixed-bed reactor optionally receives an inert stream comprising at least one among COₓ and water steam. Such additional stream is added upstream the reactor, so as to guarantee it complete mixing.

In the context of oxidative coupling of methane (OCM), hysteresis mode refers to the phenomenon where the performance or behavior of the catalyst and/or reactor depends on its previous states or conditions.

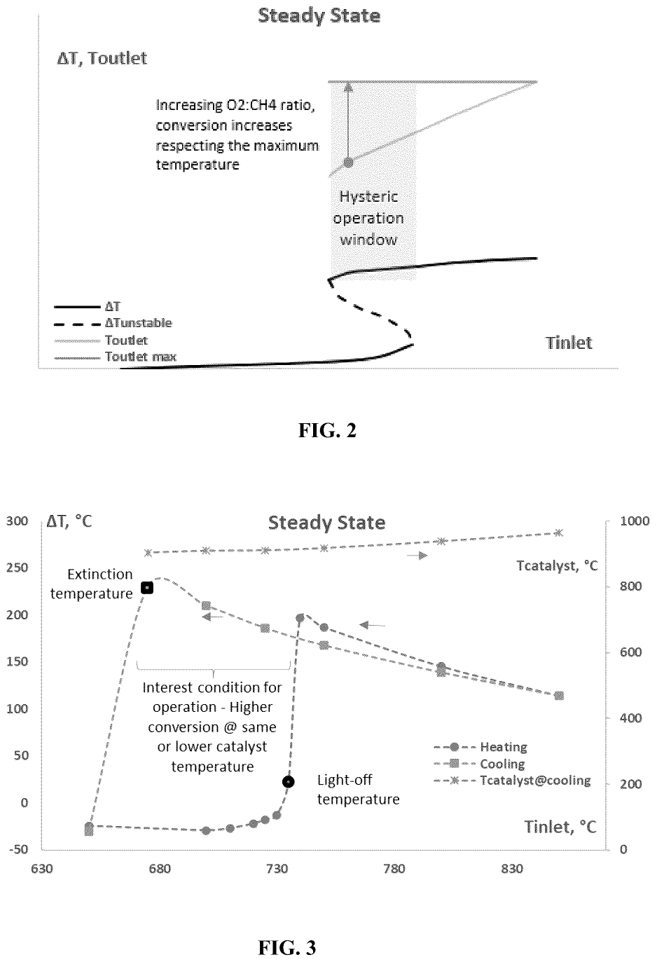

In this regard, depending on the exothermicity and the heat transfer rate between the gas and solid phases, the hysteresis phenomena may occur, as depicted by FIG. 2. The activity and design of the catalyst, catalyst bed dimension, operation condition and reactor type also affect the occurrence of the autothermic responses. Particularly, in a reactor with negligible back-mass and back-energy mixing, as in a fixed-bed reactor, heat and mass transfer constraints can be even more important.

Accordingly, the system according to the present invention is developed to provide the hysteresis behavior at adiabatic fixed-bed reactors. For that, some conditions needed to be designed and conceived to achieve the target mode.

In the system described by the present invention, the flow pattern within the reactor is assumed to follow a unidimensional plug flow model under steady-state conditions. This means that the velocity of any fluids (liquid or gas) remains constant across any cross-sectional area. The system comprises two distinct phases: a solid phase and a gaseous phase. For instance, the velocity of the fluid can typically vary between 0.02 and 10 m/s. The reactions take place on the surfaces of the solid catalyst phase, so that an exothermic reaction inside the fixed-bed containing the catalyst occurs, transferring energy between a surface of the catalyst and a stream of fluid in contact with the catalyst inside of the fixed-bed reactor. Such energy balance in the gas phase is represented by Equation (1), which can be rewritten as Equation (2), where U₂ is define in Equation (3), as the dimensionless number for convective effects in the catalyst.

$$\frac{{ρ}_{g} {u}_{z} {C}_{{p}_{g}}}{L} \frac{{dT}_{g}}{dz} = {h}_{s} {a}_{s}^{cat} ( {T}_{s} - {T}_{g} ) \qquad ( Eq . 1 )$$

$$\frac{{dT}_{g}}{dz} = {U}_{2} ( {T}_{s} - {T}_{g} ) \qquad ( Eq . 2 )$$

$${U}_{2} = \frac{{h}_{s} {La}_{s}^{cat}}{{ρ}_{g} {u}_{z} {C}_{{p}_{g}}} \qquad ( Eq . 3 )$$

wherein: ρ_{g} is the specific mass of the reaction medium in the gas phase (kg/m³), u_{z} is the surface velocity of the gas (m/s); Cₚ_{_{g}} is the heat capacity of the gas phase (J/(kg×K)); L is reactor length, T_{g} is the gas phase temperature (K); Tₛ is the solid phase temperature (K); hₛ is the bed convective coefficient (W/(m²×K)); z is dimensionless axial length of the reactor;

$${a}_{s}^{cat}$$

is the specific area of the catalyst, m²/m³.

Equations (1)-(3) represent, therefore, the variation of the flowing gas temperature due to the convective heat transfer with the catalyst (controlled by the parameter U₂).

Similarly, the energy balance equation for the solid phase at the steady state condition are represented by equations (5) to (13), as follows.

$${\sum }_{i = 1}^{NR} ( - Δ {H}_{i} ) {k}_{i} ( T ) {F}_{i} ( {C}_{j} , T ) = {h}_{s} {a}_{s}^{cat} ( {T}_{s} - {T}_{g} ) \qquad ( eq . 5 )$$

$${\sum }_{i = 1}^{NR} ( - Δ {H}_{i} ) {k}_{0 i} {e}^{- \frac{{E}_{ai}}{{RT}_{s}}} {F}_{i} ( {C}_{j} , T ) = {h}_{s} {a}_{s}^{cat} ( {T}_{s} - {T}_{g} ) \qquad ( eq . 6 )$$

$$\frac{L}{{ρ}_{g} {u}_{z} {C}_{{p}_{g}}} {\sum }_{i = 1}^{NR} ( - Δ {H}_{i} ) {k}_{0 i} {e}^{- \frac{{E}_{ai}}{{RT}_{s}}} {F}_{i} ( {C}_{j} , T ) = \frac{{h}_{s} {La}_{s}^{cat}}{{ρ}_{g} {u}_{z} {C}_{{p}_{g}}} ( {T}_{s} - {T}_{g} ) \qquad ( eq . 7 )$$

$${Ae}^{- \frac{B}{{T}_{s}}} = {U}_{2} ( {T}_{s} - {T}_{g} ) \qquad ( eq . 8 )$$

$$A = \frac{L}{{ρ}_{g} {u}_{z} {C}_{{p}_{g}}} {\sum }_{i = 1}^{NR} ( - Δ {H}_{i} ) {k}_{0 i} {F}_{i} ( {C}_{j} , T ) \qquad ( eq . 9 )$$

$$B = \frac{1}{R} {\sum }_{i = 1}^{NR} {E}_{ai} \qquad ( eq . 10 )$$

$$\frac{d}{dz} ( {Ae}^{- \frac{B}{{T}_{s}}} ) = \frac{d}{dz} [ {U}_{2} ( {T}_{s} - {T}_{g} ) ] \qquad ( eq . 11 )$$

$${Ae}^{- \frac{B}{{T}_{s}}} \frac{B}{{T}_{s}^{2}} \frac{{dT}_{s}}{dz} = {U}_{2} ( \frac{dTs}{dz} {T}_{s} - \frac{{dT}_{g}}{dz} {T}_{g} ) \qquad ( eq . 12 )$$

$$\frac{dTs}{dz} = \frac{{U}_{2}^{2} ( {T}_{s} - {T}_{g} )}{{Ae}^{- \frac{B}{{T}_{s}}} \frac{B}{{T}_{s}^{2}} - {U}_{2}} \qquad ( eq . 13 )$$

wherein: ΔHᵢ: enthalpy change, J/kg; kᵢ(T) is the intrinsic rate of the reaction; Fᵢ(Cⱼ, T) is a function that represents the reaction composition; k₀ᵢ is the reaction coefficient; E_{αi} is the activation energy of the reaction.

In view of that, the development of the hysteresis behavior can be described mathematically when the derivative of the solid phase temperature in relation to the axial length is infinite, which can be expressed by equation (14), which can provide two solutions: (i) two steady state—multiple steady states and development of extinction-ignition phenomena, or (ii) single steady state and trivial dynamic behavior.

$${Ae}^{- \frac{B}{{T}_{s}}} \frac{B}{{T}_{s}^{2}} = {U}_{2} \qquad ( eq . 14 )$$

In the limiting condition, the two steady state solutions become equal to each other and the parabolic curve does not cross the exponential curve at two points, but touches tangentially the exponential curve, so that equation (15) is fulfilled.

$${U}_{{2}_{critic}} = \frac{4 {Ae}^{- 2}}{B} \qquad ( eq . 15 )$$

In view of that, two regions of the parameter space can be defined: one that leads to the autothermic hysteresis behavior (U₂<U₂_{_{critic}}) and one where trivial single steady-state responses are located (U₂>U₂_{_{critic}}). According to the present invention, the system is designed in terms of catalyst activity, catalyst geometry, catalyst bed dimensions and feed velocity to operate in hysteresis mode, so U₂<U₂_{_{critic}}. Consequently, the catalyst provides a dimensionless number for heat transfer between the fixed-bed and the stream comprising oxygen smaller than a critical dimensionless number, so as to keep a temperature of the fixed-bed at least 50° C. higher than a temperature of the stream comprising oxygen.

Furthermore, the existence of the autoignition behavior associated with the hysteresis allow the feeding of cooled feedstocks and the consequent increase of the O₂/CH₄ mass ratio to ranges of 0.23 to 0.47 and of the reaction conversion to ranges of 11% to 26%, without surpassing the outlet temperature constraints. The autoignition behavior allows for an increased ratio of oxygen to methane in the fixed-bed reactor.

As discussed, the present invention provides a system based on a standard adiabatic reactor design, working with a catalyst that promotes a hysteresis behavior. For that, the catalyst must be active, and must have a low heat transfer coefficient, so U₂<U₂_{_{critic}}.

Therefore, it is clear that the catalyst design is one of the key points to achieve the hysteresis behavior and, therefore, better yields and efficiency.

In this regard, according to the present invention, the system is fed with a catalyst is in the form of pellets or powders with average particle size ranging from 50 μm and 40 mm.

In accordance with one aspect, the catalyst has a morphological shape selected among cylindric, hollow, spheric, multilobe and others, and a ratio between an external surface area (A) and an external volume (V) in the range of 100 m⁻¹ and 30000 m⁻¹, the external surface area and the external volume being determined as the particle-fluid heat transfer area and the total reactor bed volume. This feature, together with the other discussed features of the catalyst, is an important point to achieve the desired conditions of the system, since the external surface area relates to reaction rate and heat release, while the external volume interferes in the heat capacity. Therefore, developing suitable ratios between both physical properties leads to great heat transference capabilities aligned with appropriate reaction rates, thus providing the conditions needed to achieve the desired conditions of the system.

According to another aspect of the present invention, the catalyst has an active phase and at least one inert support, wherein a ratio between the active phase and the at least one inert support ranges from 5% to 100%. Such ratio impacts the catalyst is active, which is one of the requirements to achieve the hysteresis mode.

Moreover, the catalyst should be sufficiently active for the OCM reaction, with A and B coefficient values (Equation 9 and 10) ranging between 1.00×10² and 1.00×10⁸K and 1.00×10² and 1.00×10⁸K, respectively.

According to another embodiment, the active phase of the catalyst is selected from the group consisting of monometallic, bimetallic, trimetallic, multimetallic oxides, and mixtures thereof. Suitable catalysts are MgO, CaO, BaO, La₂O₃, SrO, TiO₂, Sm₂O₃, Nd₂O₃, Fe₂O₃, Gd₂O₃, ZrO₂, Nb₂O₅, LaAlO₃, LaGaO₃, LaInO₃, CaO—La₂O₃, Sr—La₂O₃, CeO₂—La₂O₃, Sm₂O₃/MgO, TbOx/MgO, CeO₂/MgO, Li-TbOx/n-MgO, Na—TbOx/n-MgO, La_{0.9}Ba_{0.1}InO_{3-δ}, La_{0.8}Ba_{0.2}InO_{3-δ}, La_{0.6} Ba_{0.4}InO_{3-δ}, SrTi_{0.9}Pb_{0.1}O₃, SrTi_{0.9}Mg_{0.1}O₃, SrTi_{0.9}Ca_{0.1}O₃, SrTi_{0.9}Ba_{0.1}O₃, SrTi_{0.9}Ba_{0.1}O₃, SrTi_{0.9}Al_{0.1}O₃, Sr₂Ti_{0.9}Mg_{0.1}O₄, Sr₂Ti_{0.9}Al_{0.1}O₄, Me-W—Mn/SiO₂, Li—W—Mn—O—SiO₂, Na—W—Mn—O—SiO₂.

On the other hand, according to an embodiment, the inactive phase of the catalyst is selected from the group consisting of silica, alumina, chromia, metal oxides, halogenated salts, carbon sulfide, graphite, molecular sieves, zeolites, and a mixture thereof, used to support the active phase and reduce the overall catalyst activity.

For the commercial scale, catalyst pellets can be even more favorable for development of the hysteresis behavior than catalyst powder, due to the energy transfer restriction associated with the smaller specific area for heat transfer from the solid catalyst phase to the gas phase, a necessary condition for development of hysteresis.

According to a further aspect, the disclosure provides a method for catalytic oxidative coupling of methane comprising: directing a stream comprising oxygen and methane, mixed upstream of a fixed-bed reactor containing a catalyst; or, alternatively, directing two separated streams comprising oxygen to a first inlet and methane to the second inlet of a fixed-bed reactor; catalyzing oxidative coupling of methane in a hysteresis mode; and controlling a transfer of heat and mass between the fixed-bed reactor and surroundings.

According to an embodiment, the method optionally comprises directing an inert stream comprising at least one among COx or water steam to the fixed-bed reactor.

According to another embodiment, the method further comprises cooling an effluent comprising ethylene and other hydrocarbons, hydrogen, non-converted methane, COx and water; compressing the effluent; removing acid and water from the effluent; cryogenically separating hydrogen and methane; and separating C₂ compounds from C₃₊ compounds.

The effluent is generally cooled by a sequence of heat exchangers integrated with other process streams, maximizing the energy integration, and also by direct contact with water. The effluent is then compressed using a multistage compressor, due to the high compression ratio. The acid gas is removed by a scrubber throughout the compression stages. Finally, the hydrocarbon compounds are separated and purified, requiring low-temperature distillation and hydrogenation reaction for alkynes.

In the method according to the present invention, the catalyst is in the form of pellets or powders with average particle size ranging from 50 μm and 40 mm.

In accordance with one aspect, the catalyst has a morphological shape selected among cylindric, hollow, spheric, multilobe and others, and a ratio between an external surface area (A) and an external volume (V) in the range of 100 m⁻¹ and 30000 m⁻¹, the external surface area and the external volume being determined as the particle-fluid heat transfer area and the total reactor bed volume.

The reactions take place on the surfaces of the solid catalyst phase, so that an exothermic reaction inside the fixed-bed containing the catalyst occurs, transferring energy between a surface of the catalyst and a stream of fluid in contact (and, therefore, methane) with the catalyst inside of the fixed-bed reactor.

In the method of the present invention, the catalyst provides a dimensionless number for heat transfer between the fixed-bed and the stream comprising oxygen smaller than a critical dimensionless number, so as to keep a temperature of the fixed-bed at least 50° C. higher than a temperature of the stream comprising oxygen. Moreover, due to the design of the catalyst, previously discussed, it provides an autoignition behavior.

In order to achieve the desired conditions and efficiency of the method, a mass transfer coefficient is sufficiently high to achieve a catalyst effectiveness factor higher than 0.5 to assure the catalyst effectiveness and high activity.

In accordance with another embodiment, the head loss through the fixed-bed reactor needs to be controlled above a limit. Head loss in fixed-bed reactors refers to the pressure drop that occurs as a fluid flows through the packed bed of catalyst particles. This pressure drop is caused by frictional forces between the fluid and the solid particles, as well as any obstructions or changes in flow paths within the reactor. In this regard, according to the method of the present invention, a total head loss through the fixed-bed is smaller than 1 bar.

Another critical factor is the residence time, which is the average time a molecule spends in a reactor. It's a critical factor in reactor design and performance because it influences the extent of chemical reactions and the efficiency of the process. Essentially, it tells us how long reactants stay in the reactor before being converted into products and exiting the system. In the method and system according to the present invention, the residence time is the fixed-bed reactor is smaller than 5s, preferably smaller than 2s.

In another embodiment, the stream comprising methane and oxygen is fed to the first fixed-bed reactor at a temperature smaller than 450° C., having a lower limit at room temperature. On the other hand, the inert stream comprising at least one among COx or steam is cofed to the fixed-bed reactor. Both streams are fed respecting criteria that guarantee adequate mixing with the reagents. An outlet of the fixed-bed reactor is configured to discharge the reaction products between 700° C. and 950° C., preferably between 850° C. and 930° C. The outlet stream is maintained in absolute pressures between 1 kgf/cm² and 15 kgf/cm², preferable between 5 kgf/cm² and 10 kgf/cm².

In another embodiment, the fixed-bed is configured to be at a temperature of the fixed-bed at least 50° C. higher than a temperature of the stream comprising oxygen. Moreover, the fixed-bed reactor is sized L/D<5, preferably L/D<1.

In the method according to the present invention, a flow pattern of the stream comprising oxygen and a flow pattern of the methane follow a unidimensional plug flow pattern at a steady state (i.e., velocity of the fluid is constant for any cross-sectional area) at gas hourly space velocity <60,000 h⁻¹, preferably <45,000 h⁻¹.

Furthermore, the existence of the autoignition behavior associated with the hysteresis allow the feeding of cooled feedstocks and the consequent increase of the O₂/CH₄ mass ratio to range of 0.23 to 0.47 and of the reaction conversion to 11% to 26%, without surpassing the outlet temperature constraints. The autoignition behavior allows for an increased ratio of oxygen in the stream of air to methane in the fixed-bed reactor. In this regard, a reduced inlet temperature and an increased ratio of oxygen in the stream comprising oxygen to methane in the fixed-bed reactor, limited by reaction extinction.

In another aspect of the method of the present invention, the methane conversion is higher than 11%, preferably higher than 15%. On the other hand, according to the method of the present invention, an ethylene selectivity that is above a minimum value that may depend on the economic features of the process operation, but always higher than 10 wt %, preferably higher than 50%, being determined by the carbon ratio of the 2-carbon components (C₂) formed by converted methane (C₁).

### EXAMPLES

For clear understanding of the examples presented hereinafter, some definitions are provided as follows.

### Example (1): Hysteresis Behavior

The hysteresis condition was demonstrated in the laboratory for one specific catalyst running in a fixed-bed reactor at adiabatic condition. The lab rig is a glass reactor installed inside a furnace, heated by resistance. Thermocouples are located in the furnace chamber, at the reactor outlet and inside the catalyst bed. It is assumed that inlet temperature is the same as that of the furnace chamber. AT is the difference between the catalyst bed and the inlet.

FIG. 3 illustrates an experimental trial in laboratory in a adiabatic fixed bed reactor at the following operation conditions: the catalyst is Mn—Na₂WO₄/SiO₂ mixed with SiO₂ (inert phase) at an 80:20 ratio in particles (D₅₀) with 180-250 μm; catalyst bed has 1 cm of height and 1 cm of diameter; flow rate is 150 ml/min; the stream comprises oxygen formed by a mixture of CH₄:O₂:N₂ of 2:1:1; and the inlet temperature varies from 650° C. to 850° C.

During heating, the temperature shift during the increase of the reactor inlet temperature is shown; during cooling, the temperature shift during the reduction of the reactor inlet temperature is displayed; and the catalyst temperature during the reduction of the reactor inlet temperature is shown. It is evident that the kickoff temperature differs from the extinction temperature, unequivocally illustrating the existence of hysteretic or hysteresis behavior. Furthermore, it can be observed that higher gas operation temperature eventually approaches the catalyst bed operation temperature when the feed temperature is sufficiently small.

### Example (2): High Activity Catalyst

For high activity catalyst as a bed containing 80% w of the catalyst Mn—Na₂WO₄/SiO₂ (with 2% w of Mn and 4% of Na₂WO₄/SiO₂) and 20% w of the inert SiO₂, operating on C1:O2:N2 ratio of 2:1:1 and feed velocity (u_{z}) of 5.4 m/s, in the reactor with catalyst volume of 1.3 m³ and bed length (L) of 0,445 m, the hysteresis behavior will be observed in a large range of specific area of catalyst. Considering the specific mass of the reaction medium in the gas phase (ρ_{g}), heat capacity of the gas phase (Cₚ_{_{g}}) and bed convective coefficient (hs) are 2.27 kg/m³, 2,298 J/(kg·K) and 825 W/(m²·K), respectively, and the catalyst specific area in two different shapes are 433 m²/m³ for a seven-hole cylinder and 197 m²/m³ for a dense cylinder, the parameter U₂ could be calculated, using equation 4, on 5.67 and 1,10, respectively. From experimental data, the parameters A and B (of equations (1)-(15)) were estimated on 3.86×10⁷K and 1.82×10⁴K, respectively, and U_{2critic} calculated using equation (15) is 1,150. Therefore, in both catalyst forms the U₂ are lower than U_{2critic}, then the hysteresis behavior will be observed.

### Example (3): Low Activity Catalyst

In a similar process design used in the previous examples, low activity was tested with the use of a low activity catalyst as a bed containing 20% w of Mn—Na₂ WO₄/SiO₂ (with 2% w of Mn and 4% of Na₂WO₄/SiO₂) and 80% w of the inert SiO2 (here, the higher amount of SiO2, makes the catalyst less active and therefore it does not achieve hysteresis), operating at the same condition as the example (2). The U₂ calculated using equation (4) for both mentioned catalyst forms are the same, 5.67 and 1,10, but the parameters A and B from experimental data were estimated on 9.04×10³K and 5.92×10³K, respectively, and U_{2 critic} calculated using equation (15) in 0.83. Therefore, in both catalyst forms the U₂ are higher than U_{2critic}, then the hysteresis behavior is not observed.

### Example (4a): Catalyst Design (Powder)

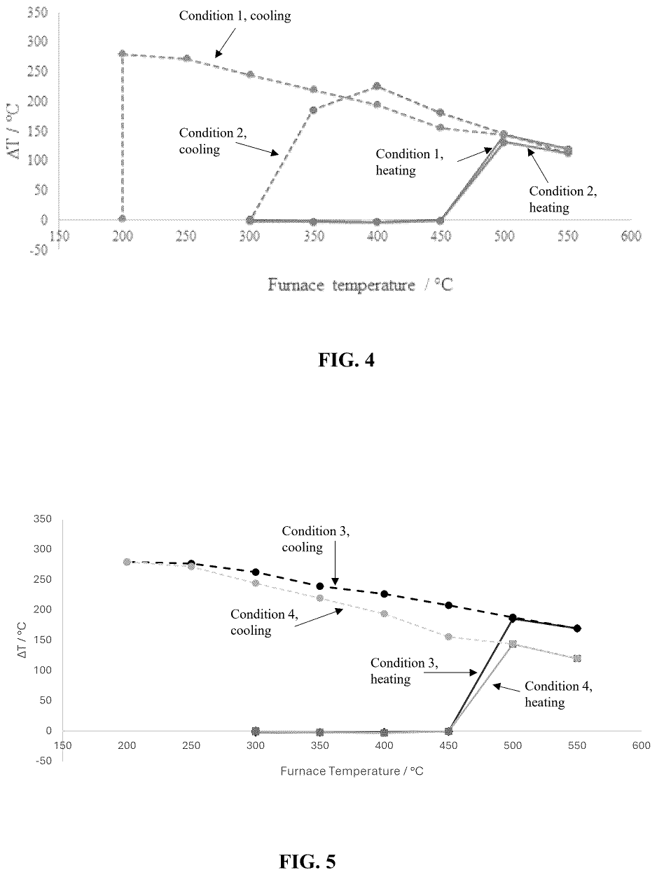

Using high activity catalyst bed (La₂O₃/CeO₂), operating on C1:O2:N2 ratio of 2:1:1 and feed velocity (u_{z}) of 0.0318 m/s, in the reactor with catalyst volume of 0.78 cm³ and bed length (L) of 1 cm, experiments were conducted varying the particle size of the catalyst into three ranges (<125 μm, 125 μm<dₚ<250 μm, dₚ>250 μm) with the objective of evaluating the effect of particle size on the hysteresis. It was observed that powders with larger particle sizes exhibit a wider hysteresis region with a lower ignition temperature, as depicted by FIG. 4 where the full lines depict heating and dashed lines represent cooling, condition 1 represents particle size above 250 μm, 0,5 g of catalyst bed, 20:80 dilution between catalyst and inert, 150 mL/min flow rate at a CH₄:O₂:N₂ ratio of 2:1:1, and condition 2 represents particle size below 125 μm, 0,5 g of catalyst bed, 20:80 dilution between catalyst and inert, 150 mL/min flow rate at a CH₄:O₂:N₂ ratio of 2:1:1.

### Example (4b): Industrial-Scale Structured Pellets

A high-activity catalyst bed comprising Mn—Na₂WO₄/SiO₂ was operated with a CH₄:O₂:N₂ (C₁:O₂:N₂) molar ratio of 2:1:1 and a superficial gas velocity u=5.4 m·s⁻¹ in a reactor with a catalyst volume=1.3 m³ and bed length L=0.445 m. Hysteresis behavior was observed over a wide span of specific catalyst surface areas. The particle-fluid heat transfer coefficient was found to depend on the catalyst particle's specific area, thereby influencing the onset and extent of hysteresis.

Under the following gas-phase properties—specific mass (ρ_{g})=2.27 kg·m⁻³, heat capacity (C_{pg})=2,298 J·kg⁻¹·K⁻¹, and bed convective coefficient (hₛ)=825 W·m⁻²·K⁻¹—the specific surface area for a seven-hole cylinder was 433 m²·m⁻³, whereas for a six-hole cylinder it was 1,072.77 m²·m⁻³. These designs led to dimensionless U₂ values of 5.67 and 23.89, respectively. The critical U₂ was calculated as 18.82, indicating that the seven-hole configuration exhibits hysteresis (U₂<U₂,crit), while the six-hole configuration does not (U₂>U₂,crit). This result demonstrates that pellet architecture (e.g., the number of internal channels/holes and the resulting specific area) directly modulates the hysteresis region through heat transfer coupling.

### Example (4c): Laboratory-Scale Pellets

A high-activity La₂O₃/CeO₂ catalyst was evaluated in a cylindrical pellet at a CH₄:O₂:N₂ ratio of 2:1:1, a superficial velocity u=0.0318 m·s⁻¹, a catalytic volume=1.57 cm³, and a bed length L=2 cm. The objective was to assess the activity of thermally pretreated, laboratory-fabricated pellets and quantify their impact on hysteresis relative to powder catalysts.

The pellet experiments exhibited a hysteresis region comparable to that observed with powdered catalyst of larger particle size (dₚ>250 μm), as illustrated in FIG. 5. In FIG. 5, solid lines denote furnace heating and dashed lines denote furnace cooling. Condition 3 corresponds to an experiment with a 0.6 g catalytic bed (0.22 g pellets+0.4 g inert) at a total flow of 150 mL·min⁻¹ and CH₄:O₂:N₂=2:1:1. Condition 4 corresponds to the powder catalyst (dₚ>250 μm) with a 0.5 g bed (with 20:80 catalyst:inert dilution), 150 mL·min⁻¹ total flow, and CH₄:O₂:N₂=2:1:1.

In all cases, the reactor outlet was directly connected to a Shimadzu 2030 gas chromatograph for continuous identification and quantification of reaction products, enabling calculation of methane (CH₄) and oxygen (O₂) conversions and C₂ product selectivity (C₂H₆ and C₂H₄).

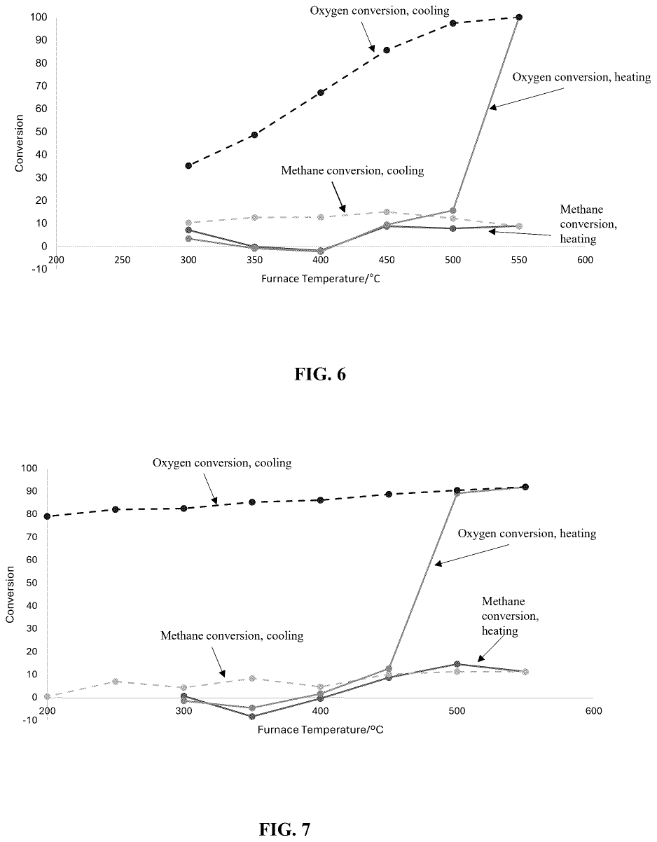

Conversion results are shown in FIG. 6 for the powder catalyst with dₚ<125 μm, and in FIG. 7 for the pellet catalyst. In both figures, oxygen conversion and methane conversion are shown during heating and cooling steps, where the solid lines indicate heating step, and dashed lines indicate cooling step.

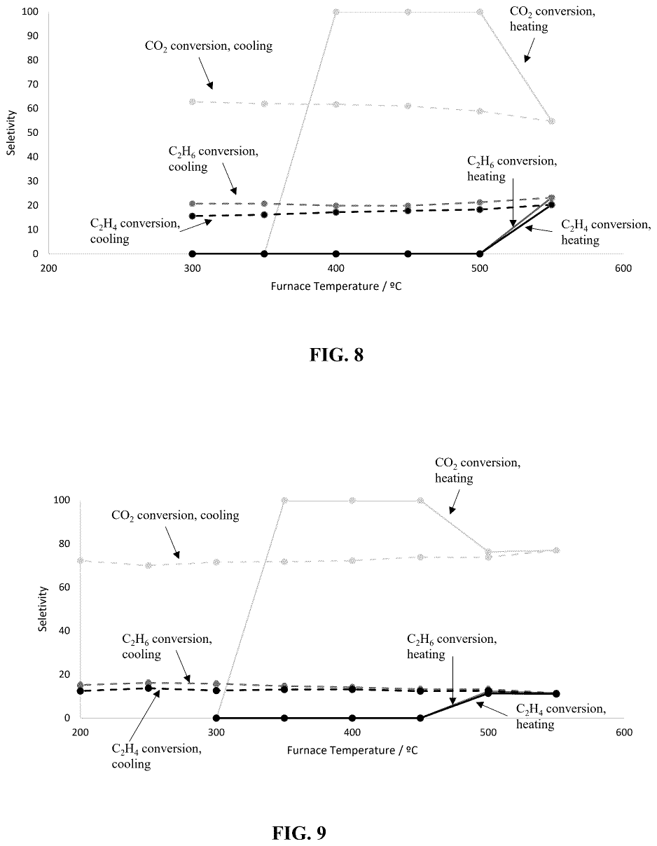

Selectivity results are presented in FIG. 8 for the powder catalyst (dₚ<125 μm) and in FIG. 9 for the pellet catalyst. In FIGS. 8-9, CO₂, C₂H₆, and C₂H₄ selectivity are shown, where solid lines indicate heating step and dashed lines indicate cooling step.

Across all data sets (FIGS. 5-9), a selectivity stability during the cooling phase is evident. Notably, for the pellet experiments, the stability window is larger, extending down to ~200° C. in the furnace, indicating enhanced thermal robustness of selectivity under decreasing temperature and supporting the potential of pelletized La₂O₃/CeO₂ designs for operational regimes that exploit hysteresis without sacrificing C₂ selectivity.

### Example (5): Catalyst Type

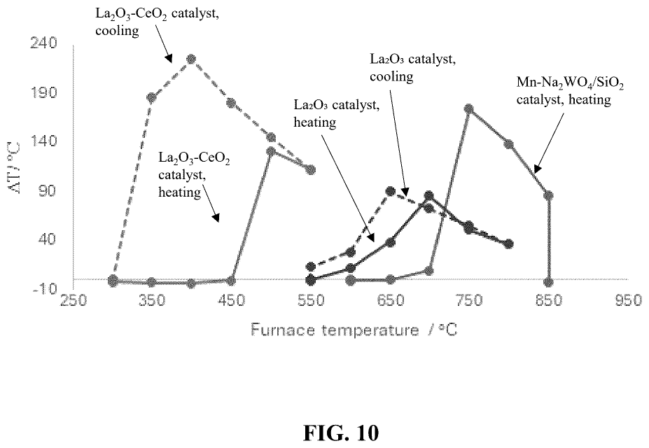

This example demonstrates the difference in hysteresis behavior for various types of catalysts, associating their activity with conversion under the same initial conditions. The catalysts considered include Mn—Na₂WO₄/SiO₂, La₂O₃/CeO₂ and La₂O₃. Among these, the La₂O₃/CeO₂ catalyst exhibits superior performance in terms of hysteresis mode. This catalyst shows a more pronounced hysteresis behavior, indicating higher activity and better conversion rates compared to the other catalysts. The variation in catalyst type significantly impacts the hysteresis region, with La₂O₃/CeO₂ providing the most favorable results as illustrated by FIG. 10, where full lines depict heating and dashed lines represent cooling. The reaction conditions are the same for all tests: particle size below 125 μm, 0.5 g of catalyst bed, 20:80 dilution between catalyst and inert, 150 mL/min flow rate at a CH₄:O₂:N₂ ratio of 2:1:1.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations may be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range may be easily recognized as sufficiently describing and enabling the same range being broken down into at least equal halves, thirds, quarters, fifths, tenths, etc. As a non-limiting example, each range discussed herein may be readily broken down into a lower third, middle third and upper third, etc. As will also be understood by one skilled in the art all language such as “up to,” “at least,” “greater than,” “less than,” and the like, include the number recited and refer to ranges which may be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A system for catalytic oxidative coupling of methane, the system comprising:
a fixed-bed reactor configured to operate at adiabatic condition and receive a stream comprising oxygen and methane; and
a catalyst contained inside of the fixed-bed reactor and configured to catalyze oxidative coupling of the methane in a hysteresis mode,
wherein the catalyst is in the form of pellets or powders with average particle size ranging from 50 μm and 40 mm, and has a morphological shape selected from the group consisting of cylindric, hollow, spheric, multilobe, and others, and a ratio between an external surface area (A) and an external volume (V) in the range of 100 m⁻¹ and 30000 m⁻¹.

2. The system of claim 1, wherein the catalyst has an active phase and at least one inert support.

3. The system of claim 2, wherein a ratio between the active phase and the at least one inert support ranges from 5% to 100%.

4. The system of claim 1, wherein the catalyst presents coefficients A and B ranging between 1.00×10² and 1.00×10⁸ K and 1.00×10² and 1.00×10⁸ K, respectively.

5. The system of claim 1, wherein the fixed-bed reactor further receives an inert stream comprising at least one among excess of methane, COₓ and water steam.

6. The system of claim 1, wherein an exothermic reaction inside the fixed-bed containing the catalyst occurs, transferring energy between a surface of the catalyst and a stream of fluid in contact with the catalyst inside of the fixed-bed reactor.

7. The system of claim 1, wherein the catalyst provides a dimensionless number for heat transfer between the fixed-bed and the stream comprising oxygen smaller than a critical dimensionless number, keeping a temperature of the fixed-bed at least 50° C. higher than a temperature of the stream comprising oxygen.

8. The system of claim 1, wherein the catalyst comprises a plurality of catalyst pellets.

9. The system of claim 1, wherein the catalyst is selected from the group consisting of monometallic, bimetallic, trimetallic, multimetallic oxides, and mixtures thereof.

10. The system of claim 1, wherein the catalyst provides an autoignition behavior.

11. The system of claim 10, wherein the autoignition behavior allows for an increased ratio of oxygen to methane in the fixed-bed reactor.

12. The system of claim 1, wherein a flow pattern in the fixed-bed reactor follows a unidimensional plug flow pattern at a steady state.

13. A method for catalytic oxidative coupling of methane, the method comprising:
directing a stream comprising oxygen and methane, mixed upstream of a fixed-bed reactor containing a catalyst;
directing two separated streams comprising oxygen to a first inlet and methane to the second inlet of a fixed-bed reactor;
catalyzing oxidative coupling of methane in a hysteresis mode; and
self-controlling a transfer of heat and mass between the fixed-bed reactor and surroundings by catalyst design.

14. The method of claim 13, wherein the catalyst is in the form of pellets or powders with average particle size ranging from 50 μm and 40 mm, and has a morphological shape selected among cylindric, hollow, spheric, multilobe and others, and a ratio between an external surface area (A) and an external volume (V) in the range of 100 m⁻¹ and 30000 m⁻¹.

15. The method of claim 13, wherein a transfer of energy occurs between a surface of the catalyst and the methane.

16. The method of claim 13, wherein the catalyst provides a dimensionless number for heat transfer between the fixed-bed and the stream comprising oxygen smaller than a critical dimensionless number, keeping a temperature of the fixed-bed at least 50° C. higher than the temperature of the stream comprising oxygen.

17.-28. (canceled)

29. A reaction product comprising a coupled methane compound, obtained by the method of claim 13.
